(19) United States
(12) Patent Application Publication
(10) Pub. No.: US 2019/0080801 A1
(43) Pub. Date: Mar. 14, 2019

(54) MEDICAL DEVICES AND SYSTEMS FOR GENERATING HEALTH RISK INFORMATION AND ALERTS BASED ON WEATHER AND ENVIRONMENTAL CONDITIONS

(71) Applicant: HEALTHEWEATHER, INC., Clifton, VA (US)

(72) Inventors: Eric J. KLOS, Clifton, VA (US); Marjorie MCGUIRK, Asheville, NC (US)

(21) Appl. No.: 16/129,109

(22) Filed: Sep. 12, 2018

Related U.S. Application Data
(60) Provisional application No. 62/558,030, filed on Sep. 13, 2017.

(51) Int. Cl. G16H 50/30

(52) U.S. Cl. CPC G16H 50/30

(57) ABSTRACT

According to embodiments, retrieved weather and environmental data from the various categories; AIR (Air Quality, Pollen) TEMP/HUM., WIND (Speed, Direction), and OUTLOOK (Cloud Cover, UV Index, and Barometric Pressure) is applied against evidence-based algorithms for estimating the increase in relative risk of onset symptoms for allergy sufferers or breathing difficulty for asthma patients based on the evidence indicating increased incidences of severe allergy impacts and asthma attacks or resultant ER visits or hospitalizations. These embodiments generate a risk index and specific impact summaries based on the increased relative risk reflected in an adjustment to the AQI and Pollen Count, which represent an estimate of the severity of the exposure, rather than the actual risk based on the patient condition. According to an embodiment, a method for generating a risk level index associated with weather-related conditions and one or more health conditions includes using a plurality of sensors, measure measuring a plurality of weather-related conditions, Determining, using the measured weather-related conditions, a risk level index associated with exposure to weather for one or more health conditions, and outputting, at a user device, an indication of the risk level index.

### RELATED APPLICATION

The present application is related to, and claims priority from, U.S. Provisional Patent Application No. 62/558,030, filed Sep. 13, 2017, entitled “MEDICAL DEVICES AND SYSTEMS FOR GENERATING HEALTH RISK INFORMATION AND ALERTS BASED ON WEATHER AND ENVIRONMENTAL CONDITIONS” to Eric J. Klos et al., the entire disclosure of which is incorporated here by reference.

### TECHNICAL FIELD

Embodiments of the subject matter disclosed herein generally relate to methods and systems for enabling personalized health risk monitoring and alert functions by correlating current weather conditions with environmental factors which tend to increase or decrease a risk that someone may experience a flare-up associated with one or more health conditions.

### BACKGROUND

Many millions of people suffer from health conditions that are impacted by weather conditions and the environment, such as allergies, asthma, migraines and joint pain among other things. For example, on hot days air pollution is typically worse than on cooler days, causing asthma triggers like ozone and smog to increase the number of flare-ups experienced by people suffering from asthma. However cold can also be troublesome for those who exercise outside, since cold can trigger coughing. Hot, dry and or windy conditions can cause increased pollen to disperse broadly in the air. Conversely, those who are allergic to pollen may prefer rainy days since these conditions tend to keep the pollen on the ground. However, those who suffer from allergies to mold may not prefer damp and humid conditions. Thus, the interactions between certain health conditions and a number of different weather and environmental conditions are both significant and complex.

Unfortunately, the technology available today to assist people in taking care of themselves based on the ever-changing weather and environmental conditions, their personal health risks and the treatment plan that they have established working with a medical professional is archaic at best. Most weather websites, like www.weather.com, etc., typically provide information that represents the severity of exposure for air quality and pollen count, but leave it to individuals to try to decide how those individual bits of weather and environmental condition information should be used to formulate a current day activity plan that will reduce an individual's likelihood of a flare-up on that particular day in his or her current location (or locations if they are travelling). Moreover, such website information makes no effort to indicate a relative risk factor for a particular malady as a function of the large number of different weather and environmental conditions that have an impact on an individual's likelihood of a flare-up.

Accordingly, it would be desirable to provide systems, methods and devices which, among other things, gather weather and environmental condition information and transform that information into a personalized risk level assessment for people who have health conditions impacted by the weather and their environment.

### SUMMARY

According to embodiments, retrieved weather and environmental data from the various categories; AIR (Air Quality, Pollen) TEMP/HUM., WIND (Speed, Direction), and OUTLOOK (Cloud Cover, UV Index, and Barometric Pressure) is applied against evidence-based algorithms for estimating the increase in relative risk of onset symptoms for allergy sufferers or breathing difficulty for asthma patients based on the evidence indicating increased incidences of severe allergy impacts and asthma attacks or resultant ER visits or hospitalizations.

These embodiments generate a risk index and specific impact summaries based on the increased relative risk reflected in an adjustment to the AQI and Pollen Count, which represent an estimate of the severity of the exposure, rather than the actual risk based on the patient condition.

According to an embodiment, a method for generating a risk level index associated with weather-related conditions and one or more health conditions includes using a plurality of sensors, measuring a plurality of weather-related conditions, determining, using the measured weather-related conditions, a risk level index associated with exposure to weather for one or more health conditions, and outputting, at a user device, an indication of the risk level.

According to another embodiment, a user device includes at least one sensor which senses a weather-related condition and outputs data associated therewith, and a processor which uses the data output from the sensor to generate a risk level associated with weather-related conditions and one or more health conditions.

According to yet another embodiment, a system includes: a plurality of sensors for measuring a plurality of different weather-related conditions and outputting data associated with the plurality of different weather-related conditions, a transceiver configured to either (a) receive other data associated with other weather-related conditions not sensed by the plurality of sensors or (b) transmit the data associated with the plurality of different weather-related conditions toward a central server, and a processor configured to either (a) generate a risk level index based on data associated with the plurality of different weather-related conditions and the other data associated with other weather-related conditions received by the transceiver or (b) to output a risk level index received by the transceiver.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 depicts a medical device according to an embodiment;

FIG. 2 depicts a medical system according to another embodiment;

FIGS. 3-4 show user interface screens according to various embodiments; and

FIG. 5 is a flow chart illustrating a method according to an embodiment.

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. The embodiments to be discussed next are not limited to the configurations described below, but may be extended to other arrangements as discussed later.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to various embodiments described herein, risks associated with weather-related health conditions are assessed based on local weather conditions and individuals are notified about a relative risk level associated with their personal health concerns and/or are provided with relevant information about remedial actions that they can take to mitigate that risk. According to one embodiment described in detail below, devices, systems and methods determine one or more current, relevant weather conditions which are local to a user of the device for allergies and/or asthma, and output risk level/impact information to that user based on the one or more relevant weather conditions. However it will be appreciated by those skilled in the in art that embodiments described herein are not limited in their application to allergies and/or asthma, but can be used to address any health condition that is impacted by the weather.

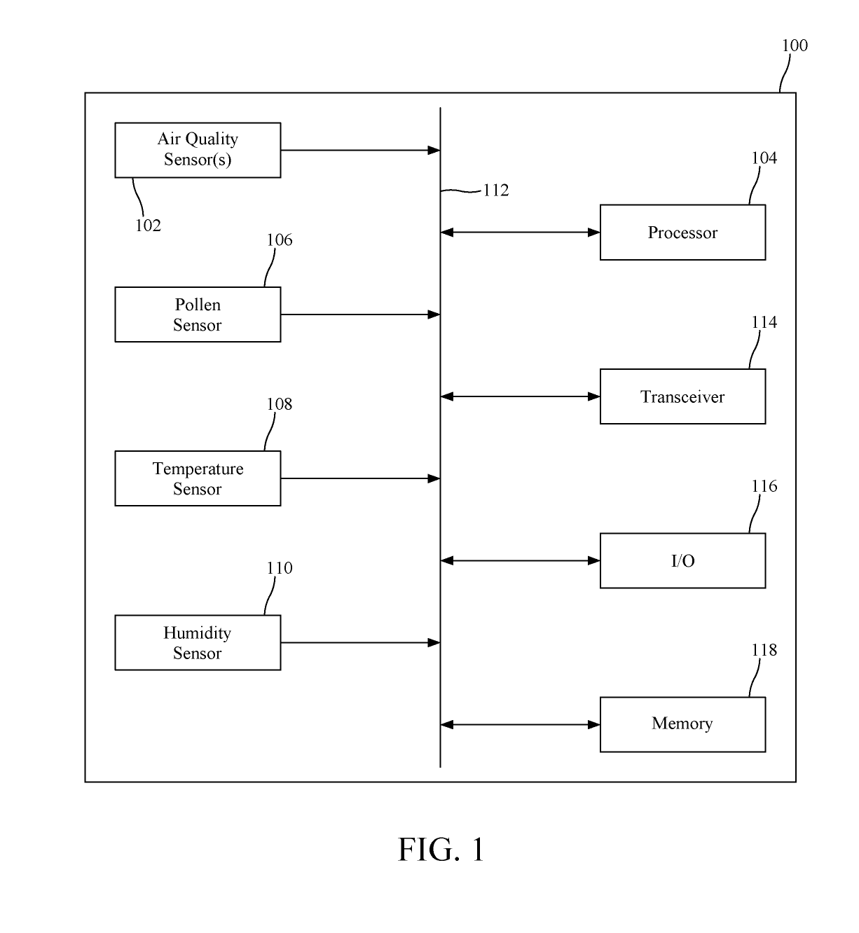

According to one embodiment, a weather-related health condition monitoring device 100 can include a number of different sensors for sensing relevant weather-related conditions, an example of which is shown in FIG. 1. For example, in this embodiment, device 100 includes one or more air quality sensor(s) 102, a pollen sensor 104, a temperature sensor 106 and a humidity sensor 108. Air quality sensor(s) 102 can, for example, measure local air pollutants such as those identified for regulation by the Clean Air Act, i.e., ground level ozone, particle pollution (also referred to herein as particulate matter or PM), carbon monoxide, sulfur dioxide and nitrogen dioxide. According to one embodiment device 100 has five air quality sensors 102, i.e., one associated with each of the pollutants identified above. Examples of such sensors and sensor technology can be found, for example, in the Air Sensor Guidebook, a publication of the Environmental Protection Agency (EPA) identified by document number EPA 600/R-14/159, published June 2014 and available at https://developer.epa.gov/air-quality-sensors/, the disclosure of which is incorporated here by reference. As will be described below the air quality data generated by air quality sensor(s) 102 can be used to issue risk level indications and/or suggested actions associated with asthma by processor 104.

Device 100 can also include a pollen sensor 106. Examples of pollen sensors which can be used as sensor 106 are described in U.S. Patent Publication No. 2007/0044579 and U.S. Patent Publication No. 2004/0066513, the disclosures of each of which are incorporated here by reference. As discussed below, the pollen data generated by the pollen sensor 106 can be used to issue risk level indications and/or suggested actions associated with allergies by processor 104. Device 100 also includes a temperature sensor 108 and a humidity sensor 110 which provide data to processor 104 regarding current temperature and humidity in the device's local area via bus or interconnect 112.

Device 100 also includes a transceiver 114, one or more input/output devices 116 and memory 118. Transceiver 114 can, for example, be a wireless transceiver which is configured to receive radio signals via one or more of a number of different standardized air interfaces, e.g., WiFi, cellular (LTE), Bluetooth, etc. According to one embodiment transceiver 114 can be used to obtain additional weather-related data to supplement that gathered by sensors 102, 106, 108 and 110 for generating risk level indications and/or other health related notifications by processor 104. According to another embodiment, the data sensed by sensors 102, 106, 108 and 110 can be transmitted to a central server (not shown in FIG. 1) for storage and subsequent usage in generating personalized alerts for the user of device 100 by using machine learning to adapt the risk level assessment for the user's own history of flare-ups. Risk level generation (examples of which are provided below) can be generated locally by processor 104 or remotely, e.g., by the central server, in which latter case they are transmitted back to device 100 for output via one of the I/O devices 116, e.g., a display or speaker. Memory 118 can be used to store various data including sensed weather-related conditions and flare-up history as described below, as well as to store software/program code which is configured to transform the sensed data into a risk level and/or other output information.

Device 100 can be housed in any desired form factor. According to one embodiment, device 100 is a wearable device, e.g., a watch or a belt mounted device. According to another embodiment, which will be described in more detail below, device 100 can be a cell phone.

There are a number of different ways in which the weather-related sensed data can be used to generate a risk level and/or other health related information according to various embodiments. Generally speaking, a risk level can be stated to be a function of the sensed weather-related parameters, i.e.:

Risk Level=f(air quality, pollen count, temperature, humidity) (1)

However other factors can also be considered, e.g., wind speed, wind direction and weather outlook or forecast. For example, wind speed and direction can impact the spread of pollen and therefore may increase (or decrease) the risk an allergic flare-up. Similarly the weather outlook, e.g., fog, rain, cold, etc., can also impact the relative risk level for an allergy or asthma attack. For example, fog, mist and may be aggravating factors for those suffering from asthma. Rain can be a benefit for those suffering from pollen related allergies. These other weather-related conditions, if not detected by sensors within device 100, can have their values signaled to device 100 via transceiver 114. Thus, according to another embodiment, risk level can be determined as a function of both directly sensed values from sensors disposed on or in device 100 and other values which are received from a central server or via an API from a website (or alternatively where the sensed values are transmitted by device 100 to another location, e.g., a central server, which also gathers the non-sensed values. In this case, the risk level can be calculated as:

Risk Level=f(air quality, pollen count, temperature, humidity, wind speed, wind direction, outlook) (2)

It will be appreciated that not all of the weather-related conditions contribute equally to the likelihood of a flare-up in a weather-related health condition, e.g., allergies and/or asthma. Instead some will contribute more to this likelihood than others. Thus, according to another embodiment, risk level can be calculated as:

Risk level=a(air quality)+b(pollen count)+c(temperature)+d(humidity)+e(wind direction)+f(wind speed)+g(outlook) (3)

where a, b, c, d, e, f, and g are weighting values which can be constants or variable functions. In some embodiments, one or more of the weighting values can be zero.

For example, an embodiment in accordance with equation (3) can be implemented using if/then statements in conjunction with evidence based values taken from the following tables.

TABLE 1
Allergy Risk Level Values and Impact Summaries

| | | | IMPACT | IMPACT | |
|---|---|---|---|---|---|
| | | | SUMMARY - | SUMMARY - | |
| | Threshold | | SHORT | LONG | DAILY |
| SUSCEPTIBILITY | or Variance | RISK | FORM | FORM | RECOMMENDATIONS |
| A uniform | “low” 0-2.4 | 1 | Prevailing | The pollen | It's unlikely you'll |
| Pollen Scale | | | Risk Factor: | count is | experience |
| | | | Pollen | low. Be | symptoms, but it |
| | | | | mindful of | doesn't hurt to have |
| | | | | the | an allergy medicine |
| | | | | predominant | with you, just in |
| | | | | pollen | case. |
| | | | | type. | |
| | “low | 2 | Prevailing | The pollen | 1) Your long-term |
| | medium” | | Risk Factor: | count is in | allergy medication |
| | 2.5-4.8 | | Pollen | the low- | may suffice for today |
| | | | | medium | 2) But, if your |
| | | | | range. If | predominant pollen |
| | | | | you are | allergen is present, |
| | | | | allergic to | you may consider |
| | | | | the | taking your short- |
| | | | | predominant | term allergy |
| | | | | pollen | medicine 30 minutes |
| | | | | type, you | before going |
| | | | | may show | outdoors. |
| | | | | some | |
| | | | | allergic | |
| | | | | symptoms | |
| | “medium” | 3 | Prevailing | The pollen | You may consider |
| | 4.9-7.2 | | Risk Factor: | count is | taking your allergy |
| | | | Pollen | medium. If | medicines 30 |
| | | | | you are | minutes before going |
| | | | | allergic to | outdoors. |
| | | | | the | |
| | | | | predominant | |
| | | | | pollen | |
| | | | | type, allergy | |
| | | | | symptoms | |
| | | | | may be | |
| | | | | more acute. | |
| | | 4 | Prevailing | The pollen | 1) Take your allergy |
| | | | Risk Factor: | count is in | medicines 30 |
| | | | Pollen | the | minutes before you |
| | | | | medium- | go outdoors. 2) If |
| | | | | high range. | you must go outside |
| | | | | If you are | consider wearing a |
| | | | | allergic to | microfiber facemask |
| | | | | the | if you will be working |
| | | | | predominant | in the yard or near |
| | | | | pollen type | your predominant |
| | | | | you will | pollen type, and 3) |
| | | | | experience | You may consider |
| | | | | increased | staying inside. |
| | | | | allergy | |
| | | | | symptoms | |
| | | | | and | |
| | | | | discomfort. | |
| | “high” 9.7-12 | 5 | Prevailing | The pollen | 1) Take your allergy |
| | | | Risk Factor: | count is | medicines 30 |
| | | | Pollen | high. If you | minutes before you |
| | | | | are allergic | go outdoors. 2) It's |
| | | | | to the | likely if you go |
| | | | | predominant | outside you will |
| | | | | pollen | experience allergy |
| | | | | type, you | symptoms |
| | | | | will be | immediately, so you |
| | | | | extremely | may wish to stay |
| | | | | susceptible | indoors. |
| | | | | to allergy | |
| | | | | symptoms | |
| | | | | when | |
| | | | | outdoors. | |
| Temperature | Temperature | 2 | Prevailing | Hot | 1) Take your allergy |
| | 22-28 C., | | Factor: Hot | temperatures | medicines 30 |
| | 72.1-82.4 F. | | Temperatures | (especially | minutes before you |
| | | | | following | go outdoors. 2) If |
| | | | | wet | you must go outside |
| | | | | weather) | consider wearing a |
| | | | | increases | microfiber facemask |
| | | | | plant, tree, | if you will be working |
| | | | | and flower | in the yard or near |
| | | | | growth and | your predominant |
| | | | | subsequent | pollen type, and 3) |
| | | | | pollen. | You may consider |
| | | | | | staying inside. |
| Humidity | High | 1 | Prevailing | High | 1) Take your allergy |
| | humidity | | Factor: | humidity | medicines 30 |
| | with high | | Humidity | (threshold | minutes before you |
| | temperatures | | | yet to be | go outdoors. 2) If |
| | (thresholds | | | conclusively | you must go outside |
| | not verified) | | | determined) | consider wearing a |
| | | | | often | microfiber facemask |
| | | | | contributes | if you will be working |
| | | | | to dense | in the yard or near |
| | | | | concentration | your predominant |
| | | | | of pollen | pollen type, and 3) |
| | | | | in | You may consider |
| | | | | atmosphere | staying inside. |
| | | | | causing | |
| | | | | greater | |
| | | | | impact. | |
| Wind | Winds > 5 m/s | 2 | Prevailing | Winds tend | On days with |
| | | | Factor: | to increase | increased winds, a |
| | | | Increased | the | microfiber mask |
| | | | Winds | dispersion | could be helpful in |
| | | | | of pollen | preventing airborne |
| | | | | spores in | pollen from being |
| | | | | the air. | breathed in. |
| | | | | This | |
| | | | | dispersion | |
| | | | | may cause | |
| | | | | you to | |
| | | | | experience | |
| | | | | increasing | |
| | | | | symptoms. | |
| Precipitation | Mist or | −1 | Prevailing | A light rain | Continue your |
| | drizzle or | | Factor: Light | over a | allergy medications, |
| | light rain | | Rain | longer | but you may |
| | | | | period | experience less |
| | | | | tends to | symptoms for now. |
| | | | | “clear” the | |
| | | | | air. Pollen | |
| | | | | spores | |
| | | | | present in | |
| | | | | the air fall | |
| | | | | to the | |
| | | | | ground. | |
| | shower | 1 | Prevailing | Despite | You should make |
| | | | Factor: | conventional | sure you take your |
| | | | Quick | wisdom | allergy medications if |
| | | | Shower | which | going outside |
| | | | | assumes | following a shower |
| | | | | rain is a | or thunderstorm. |
| | | | | dampening | |
| | | | | pollen | |
| | | | | presence, a | |
| | | | | quick | |
| | | | | shower stirs | |
| | | | | up and | |
| | | | | increases | |
| | | | | grass | |
| | | | | pollen. | |
| | thunderstorm | 1 | Prevailing | a hard rain | You should consider |
| | | | Factor: | or | taking your allergy |
| | | | Thunderstorm | thunderstorm | medications if going |
| | | | | stirs up | outside following a |
| | | | | and | shower or |
| | | | | increases | thunderstorm. |
| | | | | pollen | |
| | | | | presence in | |
| | | | | the air. With | |
| | | | | significant | |
| | | | | winds the | |
| | | | | pollen is | |
| | | | | dispersed | |
| | | | | more | |
| | | | | broadly. | |
| Ozone | 10 ppb any | 2 | Prevailing | Depending | Consider taking your |
| | particulate | | Factor: | on | allergy medications if |
| | count | | Increase in | sensitivity | you notice the ozone |
| | | | Ozone | to ozone, | is forecast to be |
| | | | | an increase | worse for today. |
| | | | | over the | |
| | | | | previous | |
| | | | | day mixes | |
| | | | | with pollen | |
| | | | | to worsen | |
| | | | | symptoms | |

TABLE 2
Asthma Risk Level Values and Impact Summaries

| | | | DAILYBREATH | | |
|---|---|---|---|---|---|
| | | | RISK | IMPACT | |
| | Threshold | | INDEX - | SUMMARY - | |
| | or | | SHORT | LONG | DAILY |
| SUSCEPTIBILITY | Variance | RISK | FORM | FORM | RECOMMENDATIONS |
| AQI - Each | “Good” | 0 | Prevailing | The air | 1) Just be mindful if |
| category | AQI is 0 to | | Factor: Air | quality is | you will be outdoors |
| corresponds to | 50 | | Quality | good. It is | outside your zip |
| a different level | | | | unlikely that | code. You may |
| of health | | | | air pollution | want to check the |
| concern. The | | | | will impact | DailyBreath Risk |
| six levels of | | | | your | Index for that |
| health concern | | | | breathing, | location. |
| and what they | | | | but | |
| mean are: | | | | remember | |
| | | | | there is | |
| | | | | always a | |
| | | | | risk | |
| | “Moderate” | 1 | Prevailing | If you are | 1) Consider limiting |
| | AQI is 51 | | Factor: Air | unusually | your outdoor activity |
| | to 100 | | Quality | sensitive to | to the early morning |
| | | | | ozone, you | hours. 2) If you go |
| | | | | may | outside, make sure |
| | | | | experience | you have your |
| | | | | breathing | quick-relief |
| | | | | difficulty. | medication with you. |
| | “Unhealthy | 2 | Prevailing | If you have | 1 & 2, plus add. 3) |
| | for | | Factor: Air | asthma, and | You may consider |
| | Sensitive | | Quality | especially, if | wearing a breathing |
| | Groups” | | | you are an | mask if you have |
| | AQI is 101 | | | older adult | one. |
| | to 150 | | | or a child, | |
| | | | | you may be | |
| | | | | at greater | |
| | | | | risk from the | |
| | | | | presence of | |
| | | | | particles in | |
| | | | | the air. | |
| | “Unhealthy” | 3 | Prevailing | If you have | 1 & 2 plus add. 3) |
| | AQI is | | Factor: Air | asthma, you | You may consider |
| | 151 to 200 | | Quality | may | wearing a breathing |
| | | | | experience | mask if you have |
| | | | | serious | one. 4) You may |
| | | | | effects | wish to consider |
| | | | | because the | staying indoors |
| | | | | air quality is | today. |
| | | | | unhealthy. | |
| | | | | At these | |
| | | | | levels even | |
| | | | | those | |
| | | | | without a | |
| | | | | respiratory | |
| | | | | condition | |
| | | | | may | |
| | | | | experience | |
| | | | | some | |
| | | | | adverse | |
| | | | | effects. | |
| | “Very | 4 | Prevailing | If the air is | 1 & 2 plus add. 3) |
| | Unhealthy” | | Factor: Air | very | You may consider |
| | AQI is 201 | | Quality | unhealthy, | wearing a breathing |
| | to 300 | | | the general | mask if you have |
| | | | | public may | one. 4) You may |
| | | | | experience | wish to consider |
| | | | | serious | staying indoors |
| | | | | health | today. |
| | | | | effects even | |
| | | | | if individuals | |
| | | | | do not have | |
| | | | | a respiratory | |
| | | | | condition. | |
| | “Hazardous” | 5 | Prevailing | A hazardous | 1) This level is |
| | AQI | | Factor: Air | level would | hazardous to your |
| | greater | | Quality | constitute | health. Unless |
| | than 300. | | | emergency | absolutely |
| | | | | conditions | necessary stay |
| | | | | and warrant | inside. 2) In these |
| | | | | health | conditions, if you |
| | | | | warnings to | must go outside you |
| | | | | the entire | should use a |
| | | | | populations | breathing mask. |
| | | | | to take | |
| | | | | precautions. | |
| Temperature | Temp | 2 | Prevailing | Extreme | 1) Consider limiting |
| | Greater | | Factor: | heat | your outdoor activity |
| | Than 90 F. | | Extreme Heat | increases | to the early morning |
| | | | | the | hours. You'll avoid |
| | | | | concentration | the hotter mid-day |
| | | | | of ozone, | 2) If you go outside, |
| | | | | coupling | make sure you have |
| | | | | with other | your quick-relief |
| | | | | pollutants to | medication with you. |
| | | | | worsen air | |
| | | | | quality and | |
| | | | | raise the risk | |
| | | | | of irritation | |
| | | | | to your | |
| | | | | airways and | |
| | | | | breathing | |
| | | | | difficulty | |
| Temperature | | 2 | Prevailing | Extreme | 1) Consider limiting |
| | | | Factor: | heat and its | your outdoor activity |
| | | | Extreme Heat | effects on | to the early morning |
| | | | | air quality | hours. You'll avoid |
| | | | | are | the hotter mid-day |
| | | | | particularly | 2) If you go outside, |
| | | | | impactful for | make sure you have |
| | | | | children with | your quick-relief |
| | | | | asthma. | medication with you. |
| Temperature | T <= 1.4 C. = | 1 | Prevailing | Extremely | 1) If you go outside |
| | 34.52 F. | | Factor: | cold | remember to keep |
| | | | Extreme Cold | temperatures | your nose and |
| | | | | can cause | mouth covered to |
| | | | | breathing | warm up the air as it |
| | | | | difficulty. | enters your lungs. |
| | | | | Extremely | 2) And, be aware |
| | | | | cold air can | when you transition |
| | | | | trigger a | from indoors to |
| | | | | rapid | outdoors to take |
| | | | | constriction | longer breaths |
| | | | | of your | allowing your |
| | | | | airways. | airways to loosen |
| | | | | Your initial | their constriction. |
| | | | | breathing | 3) Even in cold |
| | | | | when going | temperatures, |
| | | | | outdoors | remember to bring |
| | | | | may feel like | along your inhaler |
| | | | | the air is | just in case you |
| | | | | taking your | experience onset |
| | | | | breath | symptoms. |
| | | | | away. | |
| Humidity | “increased” | 4 | Prevailing | High | 1) Consider limiting |
| | or “high” | | Factor: High | humidity, | your outdoor activity |
| | humidity— | | Humidity | usually | to the early morning |
| | not | | | accompanying | hours. 2) If you go |
| | quantified | | | hot | outside, make sure |
| | | | | temperatures, | you have your |
| | | | | tends to | quick-relief |
| | | | | concentrate | medication with you. |
| | | | | particulates | |
| | | | | and pollen | |
| | | | | and be an | |
| | | | | exacerbating | |
| | | | | factor for | |
| | | | | patients with | |
| | | | | allergic | |
| | | | | asthma. | |
| Humidity | A 10% | 1 | Prevailing | An increase | 1) Consider limiting |
| | daily | | Factor: | in relative | your outdoor activity |
| | increase in | | Increase in | humidity | to the early morning |
| | relative | | Humidity | contributes | hours. 2) If you go |
| | humidity | | | to a | outside, make sure |
| | | | | worsening | you have your |
| | | | | air quality | quick-relief |
| | | | | and pollen | medication with you. |
| | | | | presence. | 3) Because the |
| | | | | | stagnant air |
| | | | | | concentrates |
| | | | | | particulates, you |
| | | | | | may want to |
| | | | | | consider wearing a |
| | | | | | mask. |
| Wind | low wind | 1 | Prevailing | Stagnant air | 1) Consider limiting |
| | levels | | Factor: | tends to | your outdoor activity |
| | <5 MPH | | Stagnant | worsen air | to the early morning |
| | | | Winds | quality and | hours. 2) If you go |
| | | | | pollen levels | outside, make sure |
| | | | | and thus, | you have your |
| | | | | create | quick-relief |
| | | | | greater risk | medication with you. |
| | | | | of breathing | 3) Because the |
| | | | | difficulty | stagnant air |
| | | | | | concentrates |
| | | | | | particulates, you |
| | | | | | may want to |
| | | | | | consider wearing a |
| | | | | | mask. |
| Outlook | fog | 1 | Prevailing | Foggy | 1) Even if conditions |
| | | | Factor: Fog | conditions | look like they will |
| | | | | usually | have no impact on |
| | | | | occur in | your breathing, you |
| | | | | early | should make sure |
| | | | | morning or | you have quick- |
| | | | | in the | relieve medication |
| | | | | evening. | with you. |
| | | | | Foggy | |
| | | | | conditions | |
| | | | | often impact | |
| | | | | asthmatic | |
| | | | | children. | |
| | | | | The dense | |
| | | | | precipitation | |
| | | | | may cause | |
| | | | | airway | |
| | | | | restriction. | |
| | | | | Foggy | |
| | | | | conditions | |
| | | | | may also | |
| | | | | contribute to | |
| | | | | a higher | |
| | | | | incidence of | |
| | | | | mold, which | |
| | | | | is an | |
| | | | | allergen for | |
| | | | | asthma | |
| | | | | patients. | |
| Outlook | mist | 1 | Prevailing | Misty | 1) Even if conditions |
| | | | Factor: Mist | conditions | look like they will |
| | | | | often impact | have no impact on |
| | | | | asthmatic | your breathing, you |
| | | | | children. | should make sure |
| | | | | The dense | you have quick- |
| | | | | precipitation | relieve medication |
| | | | | may cause | with you. |
| | | | | airway | |
| | | | | restriction. | |
| Outlook | A frontal | 2 | Prevailing | A rapid | 1) Even if conditions |
| | passage, a | | Factors: | decrease in | look like they will |
| | cold front | | Temperature, | temperature, | have no impact on |
| | | | Humidity, and | humidity, | your breathing, you |
| | | | Barometric | and BP | should make sure |
| | | | Pressure | within a few | you have quick- |
| | | | Decreases | days can | relieve medication |
| | | | | increase the | with you. |
| | | | | risk of | |
| | | | | asthma | |
| | | | | attack in | |
| | | | | susceptible | |
| | | | | individuals | |
| PM2.5 | Each 10 μg/m3 | 1 | Prevailing | An increase | 1) Consider limiting |
| | rise | | Factors: | in PM 2.5 | your outdoor activity |
| | in PM2.5 | | Increase in | levels for | to the early morning |
| | increases | | AQI (PM 2.5) | today over | hours. 2) If you go |
| | risk of ER | | over | yesterday | outside, make sure |
| | visit by 3-20% | | yesterday | presents a | you have your |
| | (rise | | | greater than | quick-relief |
| | in 24 | | | normal risk | medication with you. |
| | hours) | | | of breathing | 3) You may |
| | | | | difficulty for | consider wearing a |
| | | | | some | breathing mask if |
| | | | | asthma | you have one. 4) |
| | | | | patients | You may wish to |
| | | | | | consider staying |
| | | | | | indoors today. |
| Ozone O3 | increase in | 2 | Prevailing | Depending | 1) Consider limiting |
| | O3 levels | | FactorAir | on | your outdoor activity |
| | by 1 ppb | | Quality: | sensitivity to | to the early morning |
| | | | Increase in | ozone, an | hours. 2) If you go |
| | | | AQI, (Ozone) | increase | outside, make sure |
| | | | over | over the | you have your |
| | | | yesterday | previous day | quick-relief |
| | | | | could | medication with you. |
| | | | | present | 3) You may |
| | | | | greater risk | consider wearing a |
| | | | | of having | breathing mask if |
| | | | | breathing | you have one. 4) |
| | | | | difficulty | You may wish to |
| | | | | | consider staying |
| | | | | | indoors today. |
| PM10 | Each 10 μg/m3 | 2 | Air Quality: | An increase | 1) If you go outside, |
| | rise | | PM 10.0, a | in PM 10.0 | make sure you have |
| | in PM10 | | rise over | levels for | your quick-relief |
| | increases | | yesterday | today over | medication with you. |
| | risk of ER | | | yesterday | 2) You may |
| | visit by 8-12% | | | presents a | consider wearing a |
| | (rise | | | greater than | breathing mask if |
| | in 24 | | | normal risk | you have one. 3) |
| | hours) | | | of breathing | You may wish to |
| | Threshold | | | difficulty for | consider staying |
| | level of | | | some | indoors today. |
| | 400 | | | asthma | |
| | particles/ | | | patients | |
| | m3 | | | | |
| PM10 | heavy | 2 | Air Quality: | Increased | 1) In these |
| | dust, | | PM 10.0, a | PM 10 | conditions, unless |
| | blowing | | rise over | levels along | absolutely |
| | sand, etc. | | yesterday | with high | necessary, stay |
| | | | | winds may | inside. 2) If you |
| | | | | increase | must go outside, it's |
| | | | | your risk of | advised that you |
| | | | | a having | consider wearing a |
| | | | | breathing | mask. |
| | | | | difficulty | |

Tables 1 and 2 above can be used, according to an embodiment, to implement medical devices and systems in accordance with any of equations (1)-(3) as follows. For a user of device 100 which has both allergy and asthma health conditions, the user can initiate measurement of local weather-related conditions by actuating a measurement cycle using one of the I/O controls 116. This causes the sensors 102, 106, 108 and 110 to sense or measure their associated weather-related conditions and report their measured values to processor 104. At the same time, processor 104 in conjunction with transceiver 114 can request, e.g., from a central server, current values for those weather-related conditions which device 100 is not configured to directly measure, e.g., wind speed and direction and/or weather outlook. With all of these weather-related values in hand, processor 104 can evaluate them against Tables 1 and 2 to generate a risk level, impact summary and/or daily recommendations as follows.

According to this embodiment, the pollen count and the air quality value(s) are considered to be dominant factors in determining risk levels for the user of device 100 in the context of allergies and asthma, respectively, i.e., they are effectively weighted more highly in equation (3) than the other parameters. Accordingly, processor 104 determines an initial risk level for the user of device 100 for allergies by taking the pollen count value sensed by sensor 106 and mapping that input against the pollen count value bins found in the first 5 rows of column 2 in Table 1. In this embodiment, pollen values are considered in the context of the uniform Pollen Scale. Thus if, for example, the pollen sensor 105 output a pollen count which corresponded to a value of five on the uniform Pollen Scale, then processor 104 would determine that the risk level associated with allergies at that particular time for the predominant factor of pollen had a relative risk level value of three.

Similarly, for asthma, processor 104 would obtain an initial risk level value associated with air quality by traversing Table 2 above. Thus, for example, if the air quality sensor(s) output one or more values which corresponded to an Air Quality Index value of 250, then processor 104 would determine that the current risk level associated with the predominant factor of air quality for asthma was four. In this regard, those skilled in the art will appreciate that if individual sensors associated with the pollutants considered in the Air Quality Index are provided in device 100, then processor 104 can take those individual values and calculate the Air Quality Index value using known equations established by the EPA (see, e.g., https://airnow.gov/index.cfm?action=airnow.calculator) prior to mapping the AQU value into Table 2. Moreover, AQI values are simply an example of one popular air quality value that can be used as a reference for binning risk level values, and other air quality values (e.g., those associated with one or more of the individual pollutants in the AQI) can be used instead.

Thus according to this example, processor 104 has now determined that the initial risk level associated with allergies for the user of device 100 for the predominant risk factor of pollen has a value of three and that the initial risk level associated with asthma for the user of device 100 for the predominant risk factor of air quality has a value of four. According to this embodiment, processor 104 selects the greater initial risk level value for further processing to output a final risk level value, i.e., processor 104 selects the initial asthma risk level of four over the initial allergy risk level of three.

Further processing in this context refers to evaluating the values of the other weather-related values that impact the risk level for asthma in Table 2. In this example, as seen by the first column of Table 2, those values include values for temperature, humidity, wind, weather outlook and, if sensed or received values are available, PM2.5, Ozone and PM10. In this context PM2.5 and PM10 refer to particulate matter pollutants having a size of 2.5 micrometers in diameter and those having a size of 10 micrometers in diameter, respectively, see, e.g., the above-incorporated by reference Air Sensor Guidebook. Using the available values for each of these secondary contributors to the risk of asthma, the processor 104 can, according to this embodiment, adjust the initial risk level for asthma with a value associated with one of the secondary factors associated with air quality. More specifically, if any of the measured or received values associated with these secondary weather-related conditions has a value within the range or ranges specified in Table 2, then the largest corresponding risk level adjustment value is added to (or subtracted from) the initial risk level to generate a final risk level value.

For example, if the temperature was less than 1.4 degrees C., per Table 2, the initial risk level of four would be incremented by one to generate a final risk level of five (assuming that none of the other secondary weather-related conditions had a higher risk level adjustment associated therewith).

Similar calculations and table evaluations can be performed by processor 104 with respect to Table 1 when the risk level associated with allergies is considered to be greater than the risk level associated with asthma. Suppose, for example, that the prevailing factor for pollen count results in an initial risk level of two for allergies (which is greater than the corresponding initial risk level for asthma) and that the only secondary weather-related condition had a sensed or reported value which matched a range in Table 1 was the weather outlook for precipitation being a light rain. Since this particular factor tends to reduce the risk of an allergic fare-up, the initial risk level would be decreased by one, resulting in a final risk level of one.

The final risk level as well as, optionally, one or more of the corresponding impact summaries and/or daily recommendations can be output by processor 104 via an I/O device 116 such as a display or speaker. Another example of the operation of this embodiment is as follows.

The parameters provided n Tables 1 and 2 indicate hat high humidity (e.g., being measured as humidity over 50%) in combination with high heat (e.g., over 90 degrees) often triggers a flare-up for patients though the air quality is moderate and pollen count is low-medium. In this case, it's been determined (through evidence) that most allergy and asthma sufferers do experience increased symptoms and flare-ups, so the AQI rating of 2 and Pollen Count of 2, would be incremented by 1 for a DailyBreath Risk Index of 3. The following associated impact summary and recommendations can be output via device 100 as well for this sensed set of weather conditions.

DAILYBREATH IMPACT SUMMARY: High Humidity in combination with High Temperature and Moderate AQI and Low-Medium Pollen Count, create conditions under which you may encounter allergy symptoms or breathing difficulty during your day.

DAILY RECOMMENDATIONS: Hopefully, you have already taken your Breo Ellipto today. If not, it might be a good idea. If you are planning to be outside today, take your allergy medication 30 minutes before going outside and make sure you have your Advair inhaler with you just in case the conditions exacerbate breathing difficulty.

The foregoing embodiment of FIG. 1 provides for a device having a plurality of sensors capable of sensing weather-related conditions or parameters to generate data which is then used to algorithmically generate a health risk level indication as well as one or more explanatory outputs. However, as indicated above, it may not always be cost-effective or otherwise desirable to provide a large number of sensors in device 100. Accordingly, it was noted above, that one or more sensors could be replaced by received values that correspond to what those sensors would generate locally. For example, instead of providing pollen sensor 106 in device 100, this sensor could be omitted and transceiver 114 could request a local pollen count value from, e.g., a central server, when a risk level assessment is requested by the user of device 100. Thus, generally speaking, embodiments contemplate devices where all of the needed values are gathered by local sensors, and other devices where some of the needed values are gathered by local sensors and some are obtained from another source.

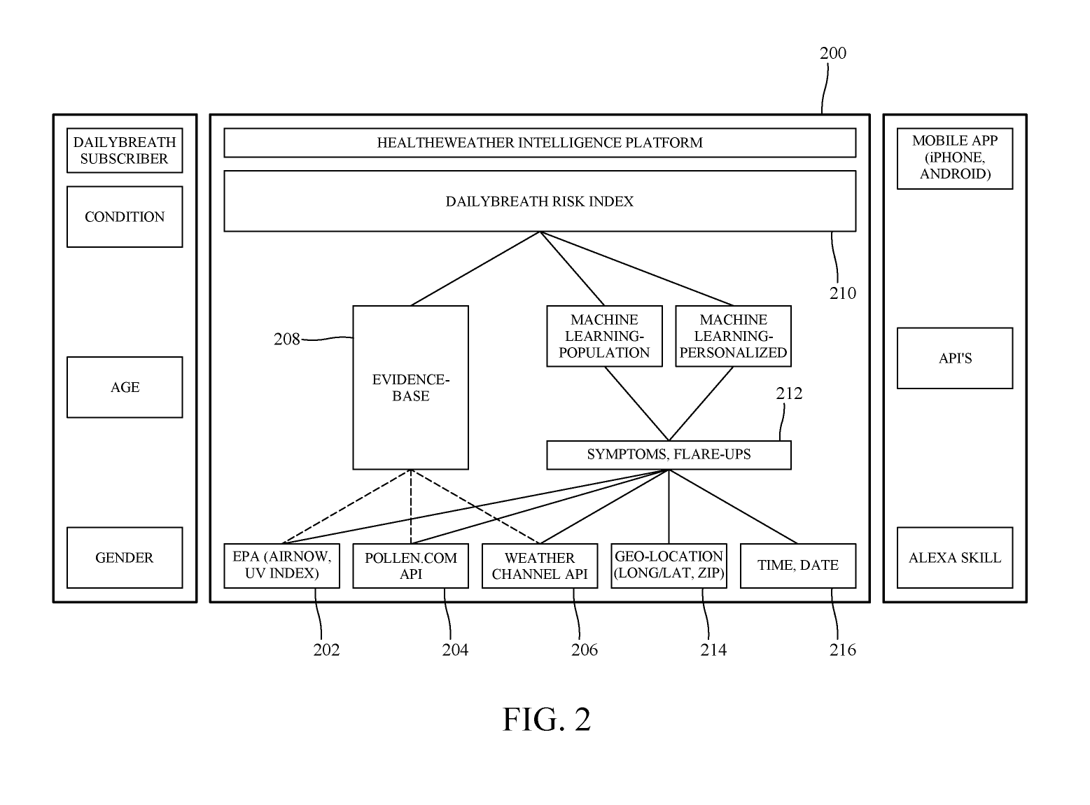

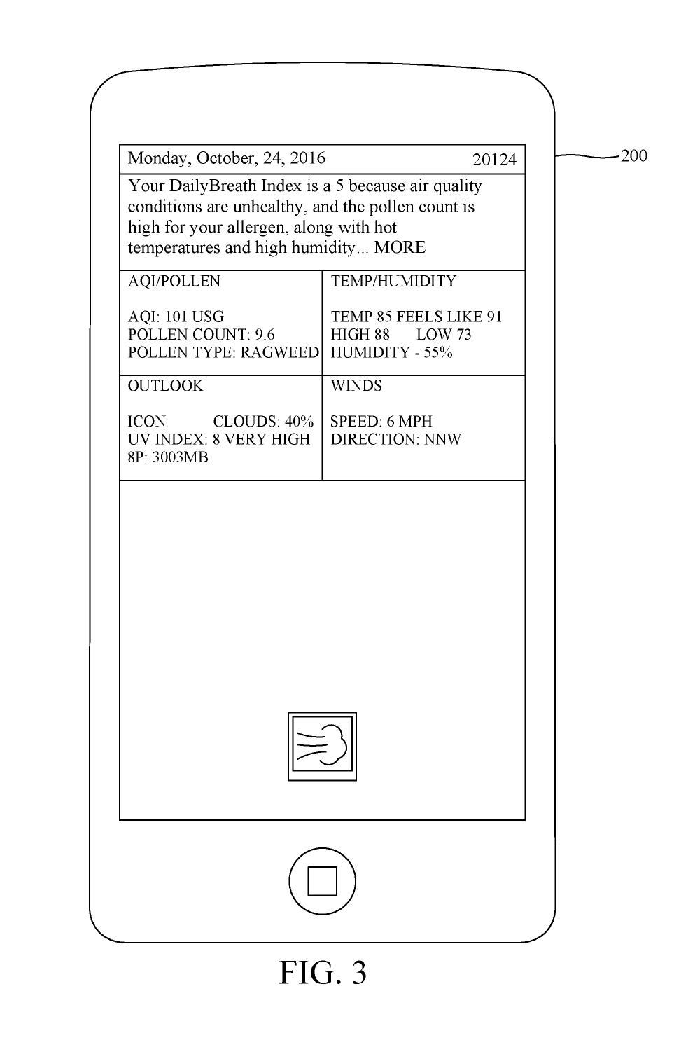

Indeed, taking this approach to it extreme, according to another embodiment, all of the values needed to cross-reference Tables 1 and 2 to generate a risk level indication and/or one or more explanatory notes can be received by the device rather than being generated by locally disposed sensors. An example of this type of embodiment is shown in FIG. 2. Therein, in device 200 the weather-related condition information is obtained via one or more application programming interfaces (APIs) 202, 204 and 206 through a transceiver from various websites (or alternatively a central server). This information can then be used to cross-reference one or more tables indicated in FIG. 2 as the evidence base 208, e.g., by a processor (not shown in FIG. 2) to generate a risk level indication or index 210 and/or explanatory outputs. Device 200 can, for example, be a cell phone (or similar device, a non-exhaustive list of which are provided below) which do not currently include the types of sensors described above with respect to the embodiment of FIG. 1. In that case, the final risk level index and associated outputs could, for example, be displayed on the screen of a cell phone 200 as shown in FIG. 3.

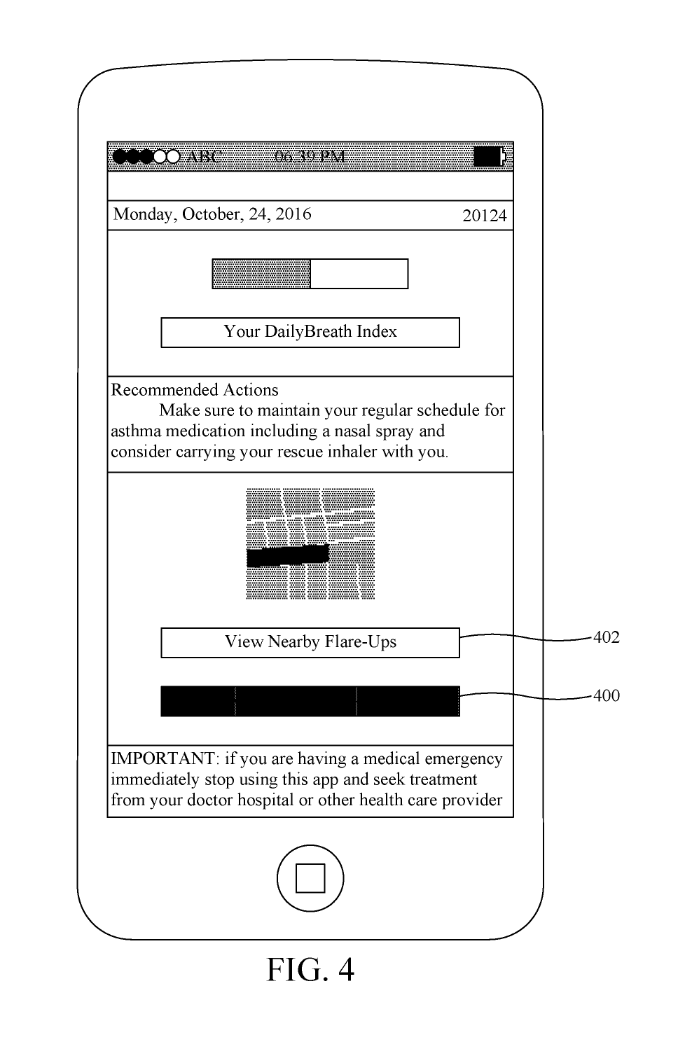

Returning to FIG. 2, this embodiment also includes another set of features which are intended to, over time, further personalize the evidence based outputs described herein. Specifically, the user of device 200 can record information associated with his or her allergy and/or asthma flare-ups (or other weather-related health conditions). For example, in the cell phone embodiment, a user interface screen such as that shown in FIG. 4 can be provided which gives the user an interface element 400 which initiates a procedure to record relevant data associated with a personal flare-up. When UI element 400 is actuated, a module 212 can collect data associated with the flare-up. For example, it can record all of the current weather-related condition data from the APIs 202, 204 and 206, as well as time and location stamping the flare-up using data from a geo-location function 214 and time/date function 216. Optionally, and additionally, symptom/flare-up module 212 can prompt the user to enter additional information associated with the flare-up, e.g., did the flare-up occur inside or outside, did the user take any measure(s) to prevent outdoor exposure impact on his or her breathing, was the user exercising when onset symptoms occurred, did the user take his or her daily and/or long term medications and/or did the user use his or her relief inhaler to help get breathing under control.

All of this flare-up information can be used to refine the evidence based/table based approach to generating risk level assessments, both on a population-wide level and on a personal/individual level using machine learning modules 214 and 216, respectively. By applying machine learning a population-health generated risk index is developed based on the empirical recording of specific weather and environmental thresholds correlating with all patient flare-ups among the condition-specific population. As flare-up data is aggregated for the population, i.e., a large number of users of devices 100 and/or 200, the system can determine the number of instances in which the various weather and environmental exposure thresholds are more or less determinant in the presence of allergy symptoms or in actual flare-ups or attacks. This information can be used to adjust the governing equations (1), (2), (3) and/or Tables 1 and 2.

Once a statistically relevant threshold is met for the individual user, then machine learning is applied to calculate a Personalized DailyBreath Risk Index. Once this Personalized Risk Index is determined then the system can begin to build alerts to inform patients when their risk is highest for experiencing exposures that could lead to a negative health outcome and make recommendations for actions to prevent these outcomes from occurring.

The near real-time empirical tracking of weather and environmental exposures related to when a patient experiences symptoms or incidences allows those exposure thresholds to be refined for individual patients. Furthermore, it provides them with personalized insights on their individual susceptibility to weather and environmental exposures. The intelligence platform described in these embodiments will deliver the where, when, and under what weather and environmental conditions, patients with specific health conditions are susceptible to negative health outcomes.

Moreover, the accumulation of historical condition-relevant daily weather and environmental exposure data on the HEALTHeWeather Intelligence Platform provides additional solution development opportunities. One example is a subscription service in which the system's stored data is combined with a specialists' patient data to give them analytics that allow them to understand the impact of weather and environmental exposures on their patient symptoms, patient inquiries, patient emergencies, and patient visits. This will improve their ability to serve their patients.

Another feature of this embodiment is the ability to provide a tracking mechanism that permits visual mapping of attacks/flare-ups and promotes community awareness of same. For example, for users that opt-in, a map interface can be provided which depicts the locations of flare-ups which occurred near to the location of a user of device 200, e.g., within a ten mile radius of the device 100, 200. This map screen can be generated by actuating button 402 shown in FIG. 4.

It should be noted that the flare-up recording and processing features, as well as the flare-up map feature described above with respect to FIGS. 2 and 4 are independent of the location of the sensors used to provide the weather-related condition information as inputs to the system, i.e., these features are equally applicable to the embodiment of FIG. 1 where some or all of the sensors are disposed within the device 100.

Additionally it should be noted that while many of the embodiments described herein indicate that the processing of the weather-related condition data to generate a risk level index and/or other associated outputs occurs at the user device, this processing could alternatively be performed at a remote location, e.g., a central server, with the results being communicated back to the user device to generate the appropriate outputs. Alternatively, some of the processing could occur at the user device 100, 200 and some of the processing could occur at a remote location. For example, the generation of a risk index for a user could occur at the user's device, whereas flare-up data storage and its machine learning processing could be performed remotely.

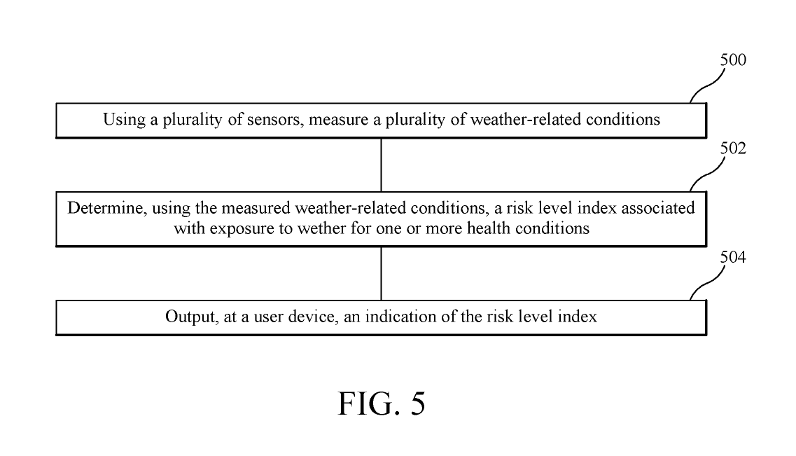

The embodiments discussed above are primarily characterized as devices and systems. However embodiments can also be characterized as methods. For example, as shown in the flowchart of FIG. 5, a method for alerting a user to a risk index associated with weather conditions and one or more health conditions can include a number of steps. A plurality of weather-related conditions can be measured at step 500. According to one embodiment, all of these measurements can be performed using sensors disposed within or on a user portable device. According to another embodiment some of these measurements can be performed using sensors disposed within or on the user device, while others of these measurements are performed remotely from the user device. According to still another embodiment, all of the measurements can be performed remotely from the user device.

At step 502, at least some of the measurements are used to determine a risk level index associated with weather conditions and one or more health conditions. Then, at step 504, the risk level index and, optionally, additional information are output by the user device.

Not all of the steps of the techniques described herein are necessarily performed in a single microprocessor or even in a single module.

Additionally, in some embodiments the non-limiting term client device or equipment is used and it refers to any type of wireline or wireless devices communicating with a network node in a cellular or mobile communication system over radio interface. Examples of client devices or user equipments (UEs) are target devices, device to device (D2D) UEs, proximity-based service (ProSe) UEs, machine type UEs or UEs capable of machine to machine communication (aka category 0 UEs, low cost and/or low complexity UEs), PDAs, iPADs, tablets, mobile terminals, smart phones, laptop embedded equipment (LEE), laptop mounted equipment (LME), USB dongles, wireless devices etc. However such devices can also include virtual reality equipments, including VR goggles, headsets, glasses and the like.

It should be understood that this description is not intended to limit the invention. On the contrary, the embodiments are intended to cover alternatives, modifications and equivalents, which are included in the spirit and scope of the invention. Further, in the detailed description of the embodiments, numerous specific details are set forth in order to provide a comprehensive understanding of the invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A method for generating a risk level index associated with weather-related conditions and one or more health conditions comprising:
using at least one sensor to measure a plurality of weather-related conditions,
determining, using the measured weather-related conditions, a risk level index associated with exposure to weather for one or more health conditions, and
outputting, at a user device, an indication of the risk level.

2. The method of claim 1, wherein the at least one sensor includes one or more air quality sensors which sense one or more of ground level ozone, particle pollution, carbon monoxide, sulfur dioxide and nitrogen dioxide.

3. The method claim 1, wherein the at least one sensor includes a pollen sensor.

4. The method of claim 1, wherein the at least one sensor includes a temperature sensor.

5. The method of claim 1, wherein the at least one sensor includes a humidity sensor.

6. The method of claim 1, wherein the step of determining further comprises:
calculating the risk level as by evaluating a function f(air quality, pollen count, temperature, and humidity).

7. The method of claim 1, wherein the step of determining further comprises:
calculating the risk level as by evaluating a function f(air quality, pollen count, temperature, humidity, wind speed, wind direction, and outlook).

8. A user device comprising:
at least one sensor which senses a weather-related condition and outputs data associated therewith; and
a processor which uses the data output from the sensor to generate a risk level associated with weather-related conditions and one or more health conditions.

9. The user device of claim 8, wherein the at least one sensor includes one or more air quality sensors which sense one or more of ground level ozone, particle pollution, carbon monoxide, sulfur dioxide and nitrogen dioxide.

10. The user device of claim 8, wherein the at least one sensor includes a pollen sensor.

11. The user device of claim 8, wherein the at least one sensor includes a temperature sensor.

12. The user device of claim 8, wherein the at least one sensor includes a humidity sensor.

13. The user device of claim 8, wherein the processor generates the risk level by evaluating a function f(air quality, pollen count, temperature, and humidity).

14. The user device of claim 8, wherein the processor generates the risk level by evaluating a function f(air quality, pollen count, temperature, humidity, wind speed, wind direction, and outlook).

15. A system comprising:
a plurality of sensors for measuring a plurality of different weather-related conditions and outputting data associated with the plurality of different weather-related conditions;
a transceiver configured to either (a) receive other data associated with other weather-related conditions not sensed by the plurality of sensors or (b) transmit the data associated with the plurality of different weather-related conditions toward a central server; and
a processor configured to either (a) generate a risk level index based on data associated with the plurality of different weather-related conditions and the other data associated with other weather-related conditions received by the transceiver or (b) to output a risk level index received by the transceiver;
wherein the plurality of sensors includes at least one air quality sensor, a temperature sensor and a humidity sensor.

16. The system of claim 15, wherein the system is part of a wearable or handheld device.

17. The system of claim 15, wherein the at least one air quality sensor senses one or more of ground level ozone, particle pollution, carbon monoxide, sulfur dioxide and nitrogen dioxide.

18. The system of claim 15, wherein the processor generates the risk level by calculating the risk level as by evaluating a function f(air quality, pollen count, temperature, and humidity).

19. The system of claim 15, wherein the processor generates the risk level by calculating the risk level as by evaluating a function f(air quality, pollen count, temperature, humidity, wind speed, wind direction, and outlook).
